(19) United States
(12) Patent Application Publication
(10) Pub. No.: US 2025/0221719 A1
(43) Pub. Date: Jul. 10, 2025

(54) IMPLANTS, INSTRUMENTATION, AND SURGICAL METHODS FOR ANKLE ARTHROPLASTY PROCEDURES

(71) Applicant: Paragon 28, Inc., Englewood, CO (US)

(72) Inventors: Dylan PAPROSKI, Littleton, CO (US); Darby MACLEISH, Denver, CO (US); Caitlin LUCAS, Littleton, CO (US); Daniel J. LEE, Denver, CO (US); Luciano Bernardino BERTOLOTTI, Denver, CO (US); Albert DACOSTA, Lone Tree, CO (US); Sven BLACHE, Wellendingen (DE)

(73) Assignee: Paragon 28, Inc., Englewood, CO (US)

(21) Appl. No.: 19/094,469

(22) Filed: Mar. 28, 2025

Related U.S. Application Data
(63) Continuation of application No. PCT/US2023/075606, filed on Sep. 29, 2023.

(51) Int. Cl. A61B 17/16; A61B 17/17

(52) U.S. Cl. CPC A61B 17/1682; A61B 17/1775

(57) ABSTRACT

An instrument includes a housing having a first end, a second end, and a body disposed therebetween. The housing also includes a first opening positioned at the first end, and a second opening positioned at the second end, with the first opening and the second opening positioned about a common longitudinal axis. The instrument also includes a first drive shaft received at least partially within the first opening and extending into the housing and a second drive shaft disposed at least partially within the housing. The instrument also includes an engagement member extending at least partially from the second opening of the housing.

### CROSS REFERENCE TO RELATED APPLICATION

This application is a continuation of International Application No. PCT/US2023/075606 filed Sep. 29, 2023, which claims priority benefit under 35 U.S.C. § 119 (e) of U.S. provisional application No. 63/377,639 filed Sep. 29, 2022, which are incorporated herein by reference in their entireties.

### TECHNICAL FIELD

The present disclosure relates to implantable medical devices, instrumentation, and surgical methodology for surgical procedures. The present disclosure relates to podiatric and orthopedic implants, instruments, and surgical methodology related the foot/ankle and/or procedures incorporating surrounding bones/soft tissue. More specifically, but not exclusively, the present disclosure relates to implants, instrumentations, and surgical methods for arthroplasty procedures of the ankle joint.

### BACKGROUND OF THE INVENTION

Many currently available implants, instruments, systems, and surgical methods for procedures involving the foot/ankle do not completely address the needs of patients. Additionally, many currently available implants, instruments, systems, and surgical methods for incorporation in procedures involving the foot/ankle, for example ankle arthroplasty procedures, fail to account for properties of joint anatomy and associated mechanical and kinematic movement patterns/capabilities.

### SUMMARY

The present disclosure is directed toward implants, instruments, and surgical methods for procedures involving the foot and/or ankle. More specifically, the present disclosure is directed implants, instruments, and surgical methods for ankle arthroplasty procedures.

One aspect of the present disclosure is directed to an instrument. The instrument includes a housing having a first end, a second end, and a body disposed therebetween. The housing also includes a first opening positioned at the first end, and a second opening positioned at the second end, with the first opening and the second opening positioned about a common longitudinal axis and at least a portion of the body of the housing being positioned substantially parallel to the common longitudinal axis. The instrument also includes a first drive shaft received at least partially within the first opening and extending into the housing, wherein at least one end of the first drive shaft terminates in a first gear member. The instrument also includes a second drive shaft disposed at least partially within the housing and includes a second gear member disposed at a first end of the second drive shaft such that the second gear member engages the first gear member, and a third gear member disposed at a second end of the second drive shaft opposite the first end of the second drive shaft. The first drive shaft includes a substantially linear geometry and the second drive shaft includes a substantially non-linear geometry. The instrument also includes an engagement member extending at least partially from the second opening of the housing and is positioned such that the engagement member is engaged by the third gear member.

In another aspect of the present disclosure provided herein, is an instrument. The instrument including a housing with a first end and a second end, a first drive shaft received at least partially within the housing, a first gear member positioned at a first end of the first drive shaft, a second gear member disposed within the first end of the housing, wherein the second gear member engages the first gear member, and a drill bit engaging the second gear member and extending out from at least a portion of the housing.

In yet another aspect of the present disclosure provided herein, is an instrument system including an instrument and a tibial trial with at least one opening. The instrument including a housing with a first end and a second end, a first drive shaft received at least partially within the housing, a first gear member positioned at a first end of the first drive shaft, a second gear member disposed within the first end of the housing, wherein the second gear member engages the first gear member, and a drill bit engaging the second gear member and extending out from at least a portion of the housing. The drill bit of the instrument is configured to rotatably pass through the at least one opening.

In a further aspect of the present disclosure provided herein, is a method of using an instrument including obtaining an instrument. The method also including performing a surgical procedure to position a tibial trial onto a tibia and inserting the instrument such that a drill bit is aligned with at least one opening in the tibial trial. The method further including using a drill to rotate the drill bit and applying a vertical translating force to at least a portion of the instrument to drill at least one opening into the tibia. In addition, the method including removing the drill bit from the tibia and the instrument from the patient.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is an elevated, front perspective view of a surgical instrument, in accordance with the present disclosure;

FIG. 2 is an alternate front perspective view of the instrument of FIG. 1, in accordance with the present disclosure;

FIG. 3 is a front, bottom perspective view of the instrument of FIG. 1, in accordance with the present disclosure;

FIG. 4 is a perspective view of an instrument system including the instrument of FIG. 1, in accordance with the present disclosure;

FIG. 5 is an alternate front perspective view of the instrument system of FIG. 4, in accordance with the present disclosure;

FIG. 6 is a perspective view of an instrument of the instrument system of FIG. 4, in accordance with the present disclosure;

FIG. 7 is an alternate perspective view of the instrument system of FIG. 4, in accordance with the present disclosure;

FIG. 8 is a side view of an instrument system, in accordance with the present disclosure;

FIG. 9 is an alternate, enlarged side view of the system of FIG. 8, in accordance with the present disclosure;

FIG. 10 is an alternate side view of the system of FIG. 8, in accordance with the present disclosure;

FIG. 11 is an alternate side view of the system of FIG. 8, in accordance with the present disclosure;

FIG. 12 is an alternate side view of the system of FIG. 8, in accordance with the present disclosure;

FIG. 13 is an elevated, rear perspective view of a portion of the system of FIG. 8, in accordance with the present disclosure;

FIG. 14 is an alternate side view of the system of FIG. 8, in accordance with the present disclosure;

FIG. 15 is an alternate elevated, rear perspective view of a portion of the system of FIG. 8, in accordance with the present disclosure;

FIG. 16 is a top view of a portion of the system of FIG. 8, in accordance with the present disclosure;

FIG. 17 is a side, cross-sectional view of a surgical instrument, in accordance with the present disclosure;

FIG. 18 is a side, cross-sectional view of a surgical instrument, in accordance with the present disclosure;

FIG. 19 is a side, cross-sectional view of a portion of the instrument of FIG. 18, in accordance with the present disclosure;

FIG. 20 is an elevated, perspective view of an instrument system, in accordance with the present disclosure;

FIG. 21 is a front view of the system of FIG. 20, in accordance with the present disclosure;

FIG. 22 is an alternate front view of the system of FIG. 20, in accordance with the present disclosure;

FIG. 23 is a perspective view of an instrument system, in accordance with the present disclosure;

FIG. 24 is a perspective view of an instrument system, in accordance with the present disclosure;

FIG. 25 is a side, cross-sectional view of a surgical instrument, in accordance with the present disclosure;

FIG. 26 is a side, cross-sectional view of a surgical instrument, in accordance with the present disclosure;

FIG. 27 is an elevated perspective view of a surgical instrument, in accordance with the present disclosure;

FIG. 28 is a side view of an instrument system, in accordance with the present disclosure;

FIG. 29 is an alternate side view of the instrument system of FIG. 28, in accordance with the present disclosure;

FIG. 30 is an alternate side view of the instrument system of FIG. 28, in accordance with the present disclosure;

FIG. 31 is a perspective view of the instrument system of FIG. 28, in accordance with the present disclosure;

FIG. 32 is a perspective view of an instrument that may be implemented in conjunction with the system of FIG. 28, in accordance with the present disclosure;

FIG. 33 is a perspective view of the instrument of FIG. 32 implemented in conjunction with a portion of the instrument system of FIG. 28, in accordance with the present disclosure;

FIG. 34 is a perspective view of an instrument implemented in conjunction with the instrument of FIG. 32 and a portion of the instrument system of FIG. 28, in accordance with the present disclosure;

FIG. 35 is a perspective view of an instrument system, in accordance with the present disclosure;

FIG. 36 is a partially transparent perspective view of the system of FIG. 35, in accordance with the present disclosure;

FIG. 37 is an alternate partially transparent perspective view of the system of FIG. 35, in accordance with the present disclosure;

FIG. 38 is an alternate partially transparent cross-sectional view of the system of FIG. 35, in accordance with the present disclosure;

FIG. 39 is an exploded view of a portion of an instrument system, in accordance with the present disclosure;

FIG. 40 is a top view of a portion of the instrument system of FIG. 39, in accordance with the present disclosure;

FIG. 41 is a side view of an implant and instrument system, in accordance with the present disclosure;

FIG. 42 is an alternate side view of the implant of the system of FIG. 41, in accordance with the present disclosure;

FIG. 43 is an alternate side view of the system of FIG. 41, in accordance with the present disclosure;

FIG. 44 is an exploded, perspective view of an instrument of the system of FIG. 41, in accordance with the present disclosure;

FIG. 45 is a side view of an alternate embodiment of an implant of the system of FIG. 41, in accordance with the present disclosure;

FIG. 46 is an alternate side view of the implant of FIG. 45 of the system of FIG. 41, in accordance with the present disclosure;

FIG. 47 is a bottom view of a portion of the implant of the system of FIG. 45, in accordance with the present disclosure;

FIG. 48 is a front view of an implant system, in accordance with the present disclosure;

FIG. 49 is an exploded, schematic front view of a portion of the system of FIG. 48, in accordance with the present disclosure;

FIG. 50 is an elevated perspective view of a portion of the system of FIG. 48, in accordance with the present disclosure;

FIG. 51 is an alternate front view of the implant system of FIG. 48, in accordance with the present disclosure;

FIG. 52 is a perspective view of an implant and instrument system, in accordance with the present disclosure;

FIG. 53 is a perspective view of an implant of the system of FIG. 52, in accordance with the present disclosure;

FIG. 54 is an alternate perspective view of the implant of FIG. 53 of the system of FIG. 52, in accordance with the present disclosure;

FIG. 55 is an elevated perspective view of an implant, in accordance with the present disclosure;

FIG. 56 is a side view of the implant of FIG. 55, in accordance with the present disclosure;

FIG. 57 is a side view of an alternate embodiment of the implant of FIG. 56, in accordance with the present disclosure;

FIG. 58 is a perspective view of an instrument, in accordance with the present disclosure;

FIG. 59 is a side view of the instrument of FIG. 58, in accordance with the present disclosure;

FIG. 60 is a perspective view of a portion of the instrument of FIG. 58, in accordance with the present disclosure;

FIG. 61 is a perspective view of an instrument, in accordance with the present disclosure;

FIG. 62 is a perspective view of an instrument, in accordance with the present disclosure;

FIG. 63 is a perspective view of an instrument, in accordance with the present disclosure;

FIG. 64 is a perspective view of an instrument, in accordance with the present disclosure;

FIG. 65 is a perspective view of an instrument, in accordance with the present disclosure;

FIG. 66 is a perspective view of the instrument of FIG. 65, in accordance with the present disclosure;

FIG. 67 is an elevated perspective view of an implant, in accordance with the present disclosure;

FIG. 68 is a perspective view of an instrument system, in accordance with the present disclosure;

FIG. 69 is a side perspective view of an instrument system, in accordance with the present disclosure;

FIG. 70 is a perspective view of a portion of the instrument system of FIG. 69, in accordance with the present disclosure;

FIG. 71 is a side, cross-sectional view of an implant system, in accordance with the present disclosure;

FIG. 72 is a side, cross-sectional view of an implant system, in accordance with the present disclosure;

FIG. 73 is a side, cross-sectional view of an implant system, in accordance with the present disclosure;

FIG. 74 is a side, cross-sectional view of an implant system, in accordance with the present disclosure;

FIG. 75 is a side view of an implant system, in accordance with the present disclosure; and

FIG. 76 is an exploded view of the implant system of FIG. 75, in accordance with the present disclosure.

### DETAILED DESCRIPTION FOR CARRYING OUT THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in U.S. Pat. No. 10,117,749, issued on Nov. 6, 2018 and entitled Subtalar Joint Implant; European Patent No. 3756626 issued on Dec. 30, 2020 and entitled Subtalar Joint Implant, European Patent Application No. 15770960.1A filed on Jul. 15, 2020 and entitled Subtalar Joint Implant; U.S. Provisional Patent Application No. 63/155,100 filed on Mar. 1, 2021 and entitled Methods for Performing Arthroplasty of the Subtalar Joint; U.S. Provisional Patent Application No. 63/167,965 filed on Mar. 30, 2021 and entitled Orthopedic Implants and Methods; International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615, filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; and/or U.S. patent application Ser. No. 16/672,505 filed Nov. 3, 2019 and titled Talus Formational And Implantation Method; and/or International PCT Application No. PCT/US2021/046920 and titled Whole Talus Implant and Method; and/or International PCT Application No. PCT/US2021/047117 and titled Implant for Focal Talus Defects and Method; which are hereby incorporated herein by reference in their entireties.

Referring to the drawings included herein, implants, instruments, systems, and surgical methods are shown and described. It should be understood that one or more of the implants, instruments, systems, and/or surgical methods shown and described herein may be implemented in conjunction with one or more of the other various implants, instruments, systems, and surgical methods shown and described herein. For example, various instruments and/or surgical methods may be implemented in order to implant one or more of the various implants/implant systems shown and described herein. Further, it should be understood that the implants, instruments, systems, and surgical methods shown herein—as well as components thereof—may be duplicated, eliminated, or otherwise combined/modified. For example, a drive shaft of an instrument may be implemented in conjunction with a power source from another instrument system in order implant one or more of the implants and/or implant system shown and described herein.

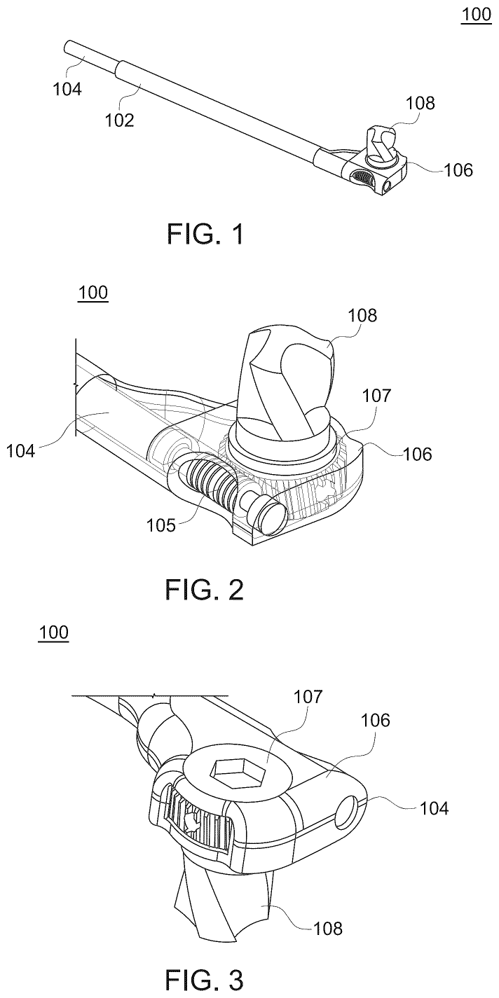

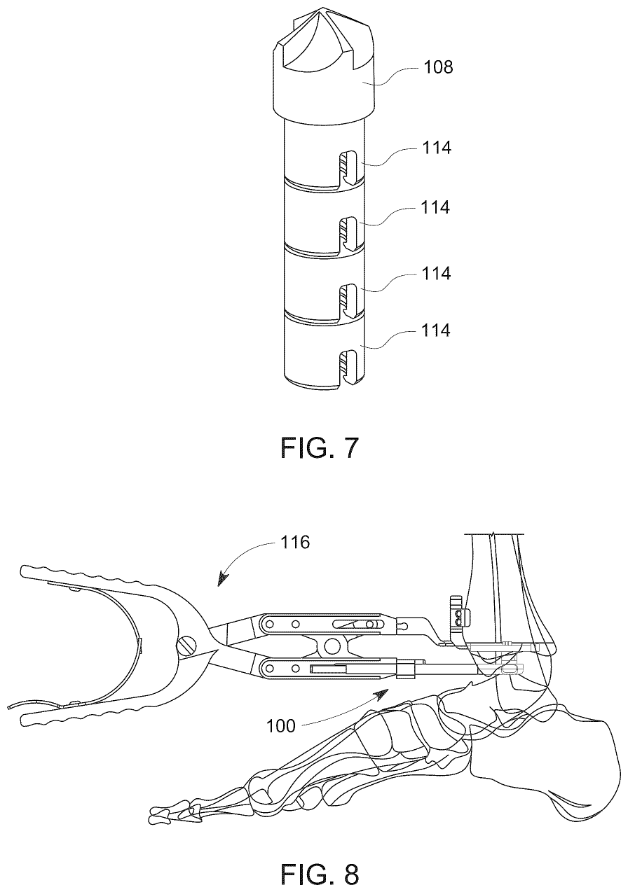

Referring now to FIGS. 1-7, and instrument 100 and components configured to be implemented in conjunction with and/or releasably couple with the instrument 100 are shown, according to an exemplary embodiment. The instrument 100 is shown to include an outer shaft 102 which may be, at an end of the outer shaft 102, integral or coupled with a housing 106. The outer shaft 102 may have a substantially cylindrical geometry and is shown to accommodate at least a portion of a drive shaft 104 at least partially therewithin and therethrough. The drive shaft 102 is shown to include a worm 105 disposed at or near a terminal end thereof, and may further be configured to be coupled with a power source or other instrument at an opposite end from the worm 105. In some aspects, other geared configurations may be implemented instead of or in addition to the worm 105, for example a miter gear and/or a series of spur gears (or other mechanisms configured to accommodate a substantially orthogonal angle). The worm 105 is shown to be disposed at least partially within the housing 106 such that the worm 105 is positioned adjacent a spur gear 107 (with the worm 105 and spur gear 107 collectively forming a worm gear). The spur gear 107 may engage with the worm 105 such that rotation of the drive shaft 104 (and thus rotation of the worm 105) actuates the spur gear 107 via engagement with the worm 105. The instrument 100 is further shown to include a drill bit 108 which may be integral with and/or coupled with a surface of the spur gear 107 (e.g., approximately 90-degrees from the teeth/engagement elements of the spur gear 107). The drill bit 108, which is shown to extend through and opening in the housing 106 and is positioned there above may be actuated by rotation of the drive shaft 104. As shown in at least FIGS. 2-3, at least a portion of the worm 105 and the spur gear 107 are positioned adjacent openings in or extend at least partially outside of the housing 106.

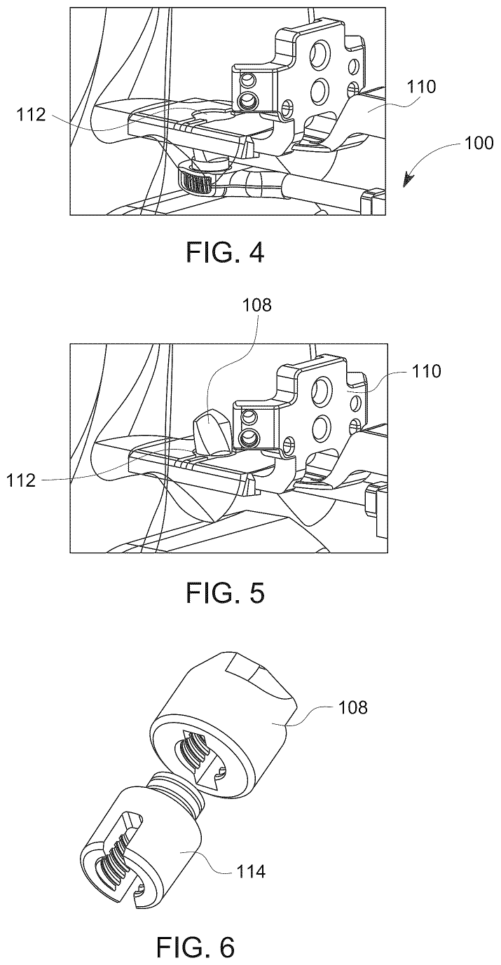

Referring to FIGS. 4-5, the instrument is shown to be implemented in conjunction with a tibial trial instrument 110. The tibial trial 110 may have one or more geometries, components, and/or dimensions the same as and/or similar to those incorporated by reference herein. The tibial trial 110 is shown to include an opening 112 in a central portion thereof configured to receive at least a portion of the drill bit 108 such that a distal portion of a tibia of a patient may be drilled with the tibia trial 110 in place (e.g., positioned adjacent the tibia). The instrument 100 may be releasably coupled with one or more modular components 114 in order to extend the drill bit 108 (which also releasably couples with the modular components 114) in a proximal direction such that more proximal portions of the tibia may be drilled while keeping the instrument 100 in a static position. The modular components 114 may be coupled incrementally by a physician such that, using the instrument 100, incrementally more proximal regions of the tibia may be drilled.

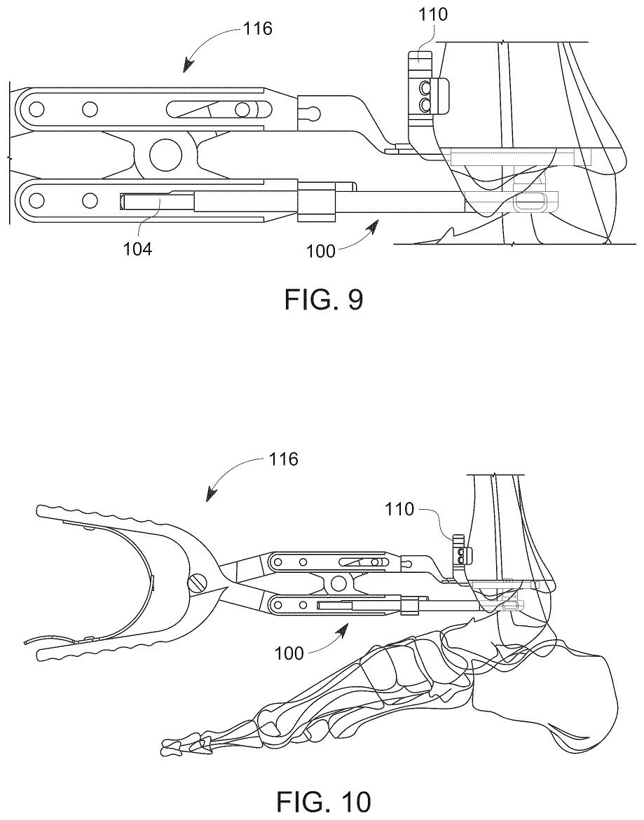

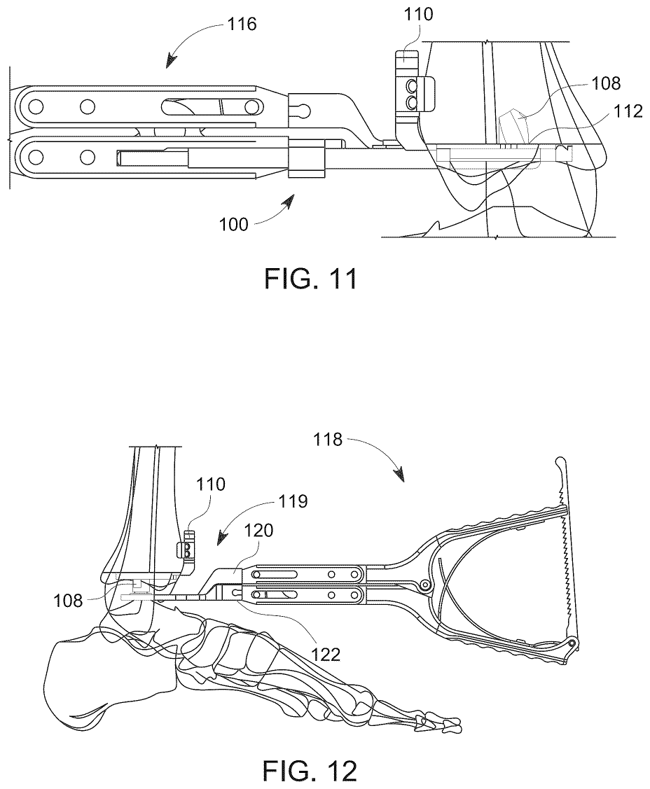

Referring now to FIGS. 8-11, an instrument system is shown including a distractor 116 and the instrument 100. As shown, the instrument 100 is disposed within a portion of the distractor 116 such that the distractor 116 may be used by a physician in order to apply a vertical, upward force to the drill bit 108 while drilling the distal tibia from outside the footprint of the tibia. For example, in some aspects the distractor 116 may be configured to apply a force and/or manipulate a component at a distal end thereof in an upward or downward direction when actuated by a physician. In some aspects, a power source (e.g., powered driver) may be coupled with the instrument 100 and/or the distractor 116 so as to translate power to the instrument 100 and thus rotate the drive shaft 104. In some aspects, the distractor 116 may be implemented in conjunction with both the instrument 100 and the tibial trial 110.

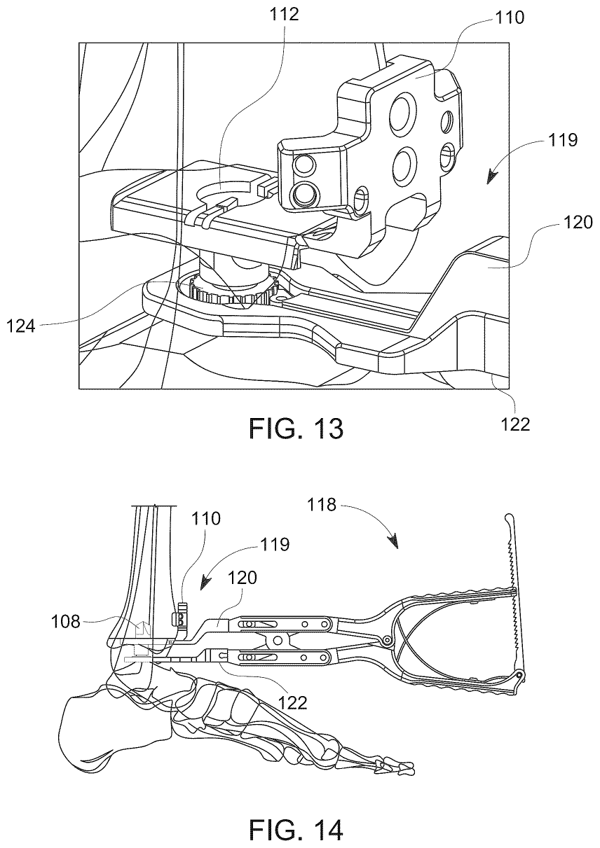

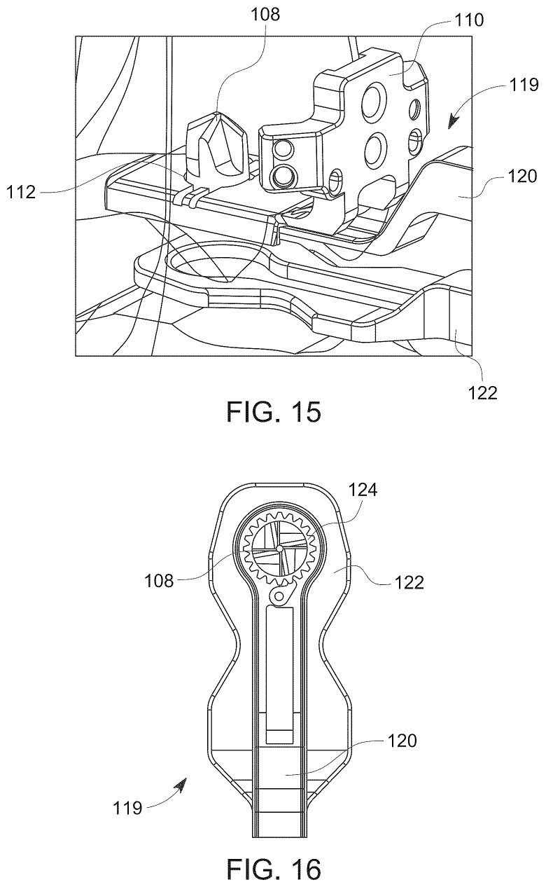

Referring now to FIGS. 12-16, an instrument system including a distractor 118 is shown. Similar to the distractor 116, the distractor 118 may be configured to receive power via coupling with a power source and/or translate power to the drill bit 108. The distractor 118 and other components shown in FIGS. 12-16 may also be implemented in conjunction with one or more components shown in FIGS. 1-11 (e.g., the tibial trial 110). The distractor 118 is shown to be releasably coupled with a paddle 122 configured to contact a talus of a patient, and an engagement element 120 including a rachet instrument 119 (e.g., including a ratchet 124, which is integral or releasably coupled with the drill bit 108) configured to be positioned adjacent a distal tibia of a patient. The ratchet 119 is shown to include the drill bit 108 positioned on an upper surface thereof, and further the ratchet 119 is shown to be positioned adjacent the tibial trial 110 such that the drill bit 108 may extend through the opening 112 and contact the distal tibia. The distractor 118 and other components of FIGS. 12-16 may be implemented without a power source, which is to say that the ratchet 119 is configured to accommodate powerless drilling of the distal tibia when used in a common ratcheting motion (e.g., moved along an arced path in a single plane).

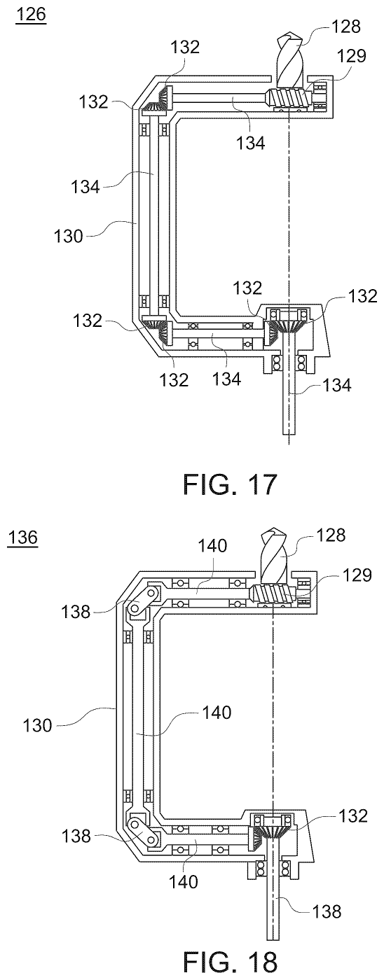

Referring now to FIG. 17, an instrument 126 is shown, according to an exemplary embodiment. The instrument 126 includes a housing 130 which, as shown, is substantially C-shaped (or U-shaped). The instrument 126 further includes an opening at both a first end and a second end of the housing 130, with said first end shown to receive a shaft 134 (e.g., a drive shaft) at least partially therein. The shaft 134 is shown to terminate in a miter gear 132 at at least one end, where the end opposite the miter gear 132 may be couplable with a power source (in some aspects, the miter gear 132 may be replaced and/or supplemented with additional gearing). The housing 130 is further shown to include three additional shafts 134, with two of said shafts 134 terminating in spur gears 132 at both ends and a third of said shafts 134 terminating with a miter gear 132 at a first end and a worm gear 129 at a second end, with said worm gear 129 positioned adjacent an opening at a second end of the housing 130. The instrument 126 is further shown to include a drill bit 128 (which may be the same as and/or similar to the drill bit 108) positioned at least partially within the housing 130 and extending partially from the second opening such that a bottom portion of the drill bit 128 may engage with the worm gear 129. Accordingly, by applying a force to the shaft 134 extending from the first opening in the housing 130 (e.g., coupling with a power source, etc.), the first shaft 134 (and corresponding miter gear 132) may be rotated, with said rotation contacting one of the spur gears 132 to drive rotation of the second shaft 134. The rotation of the second shaft 134 and corresponding spur gears 132 will in turn, through engagement with adjacent spur gears 132, drive rotation of the third shaft 134. The rotation of the third shaft 134 and spur gears thereof subsequently drives rotation of the fourth shaft 134 (with the worm gear 129) and thus drives rotation of the drill bit 128. Additionally, the instrument 126 and the housing 130 thereof may be modified such that a force (e.g., impaction, etc.) may be applied to the instrument in the direction of the drilling.

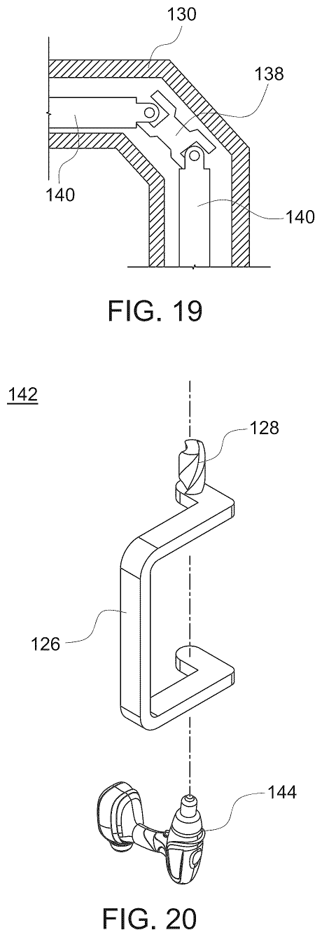

Referring now to FIGS. 18-19, an instrument 136 is shown, according to an exemplary embodiment. The instrument 136 includes the housing 130 which, as shown, is substantially C-shaped (or U-shaped) just as shown in FIG. 17. The instrument 136 further includes an opening at both a first end and a second end of the housing 130, with said first end shown to receive a shaft 140 (e.g., a drive shaft) at least partially therein. The shaft 140 is shown to terminate in a miter gear 132 at at least one end, where the end opposite the miter gear 132 may be couplable with a power source. The housing 130 is further shown to include three additional shafts 140, with two of said shafts 140 terminating at a coupling 138 at a single end, and one of said shafts 140 terminating at a coupling 138 at both ends. Of the shafts 140 that terminate in a coupling 138 at a single end, a first shaft 140 terminates at one end in a spur gear 132 that engages with the shaft 140 protruding from the housing 130 and a second shaft 140 terminates in the worm gear 129 adjacent the second opening of the housing. The instrument 126 is further shown to include a drill bit 128 (which may be the same as and/or similar to the drill bit 108) positioned at least partially within the housing 130 and extending partially from the second opening such that a bottom portion of the drill bit 128 may engage with the worm gear 129. Accordingly, by applying a force to the shaft 134 extending from the first opening in the housing 130 (e.g., coupling with a power source, etc.), the first shaft 134 (and corresponding miter gear 132) may be rotated, with said rotation contacting one of the spur gears 132 to drive rotation of the second shaft 134. The rotation of the second shaft 134 and corresponding spur gears 132 will in turn, through engagement with adjacent spur gears 132, drive rotation of the third shaft 134. The rotation of the third shaft 134 and spur gears 132 thereof subsequently drives rotation of the fourth shaft 134 (with the worm gear 129) and thus drives rotation of the drill bit 128. Additionally, the instrument 126 and the housing 130 thereof may be modified such that a force (e.g., impaction, etc.) may be applied to the instrument in the direction of the drilling.

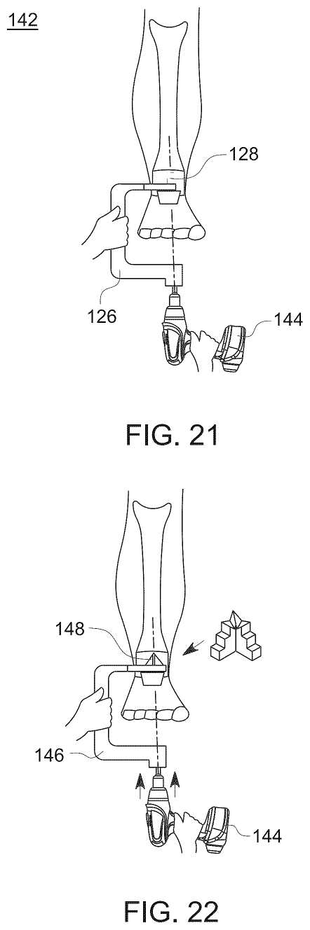

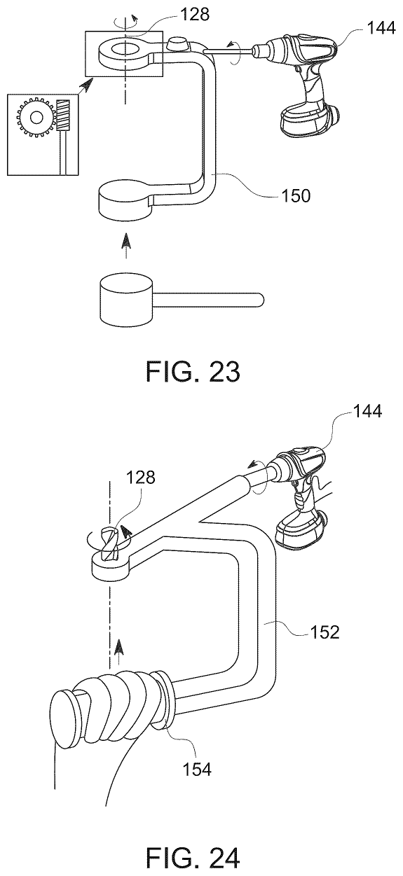

Referring now to FIGS. 20-21, an instrument system 142 (“system 142”) is shown, according to an exemplary embodiment. The system 142 is shown to include the instrument 126 which is releasably coupled with a power source 144. The power source 144 may be configured to apply a force to at least a portion of the instrument 126, for example the shaft 134, thus rotating the shaft 134 and actuating other components of the instrument 126 (including the drill bit 128). Similarly, the system 142 may be modified to include the instrument 136 as shown and described previously herein, with said power source 144 implemented in the same or similar fashion. With reference to FIG. 22, the system 142 has been modified to include an instrument 146 (which may include one or more of the same/similar components, geometries, or other features/attributes of the instruments 126, 136). The instrument 146 is shown to include a keel punch 148 in place of the drill bit 128, with reference to the instruments 126, 136. Further, the power source 144 is shown to be an impact driver (e.g., hammer drill, etc.) configured to drive the keel punch 148 into the distal tibia. However, in the instance of the system 142 (and instruments 126, 136, and others mentioned herein), the power source 144 may be configured to apply rotational force, torque, etc. configured to rotate a shaft/drive shaft or other element. With reference to FIGS. 23-24, instruments 150, 152 are shown in systems similar to that shown in FIG. 22. However, as shown in FIGS. 23-24, the instruments 150, 152 (which may be the same and/or similar to instruments 126, 136, 146) may receive a force that is manually applied to an end thereof (for example, an impaction force from a mallet or a constant force applied to a handle 154) which drives the drill bit 128 into the distal tibia while the power source 144 simultaneously applies a force to the instrument so as to rotate the drill bit 128.

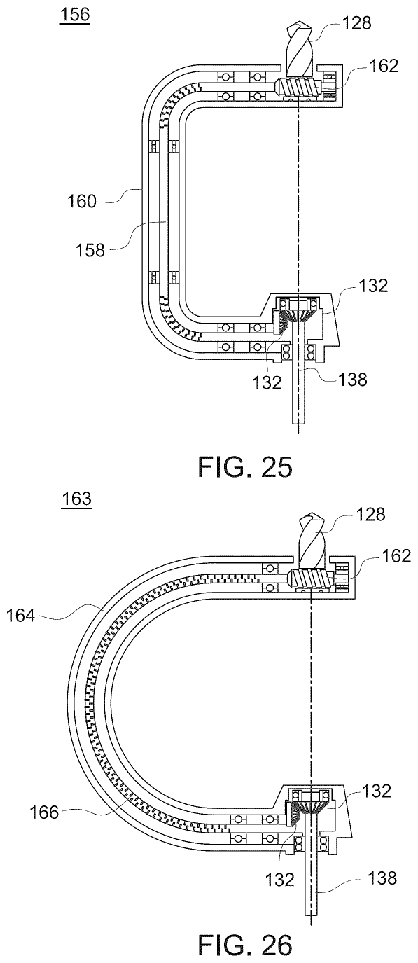

Referring now to FIG. 25, an instrument 156 is shown, according to an exemplary embodiment. The instrument 156 is shown to have a geometry that may be the same as and/or similar to the geometry of the instrument 126 and/or the instrument 136 as shown and described previously herein. The instrument 156 is shown to include a housing 160, with the housing 160 including an opening at opposite ends thereof. As shown, the first and second openings of the housing 160 are configured about a common longitudinal axis, and it should be understood that such arrangement of the first and second openings in the housing 160 may be characteristic of other instruments shown and described herein, for example the instruments 126, 136, etc. However, such arrangement of the first and second openings of the housing may also be about independent longitudinal axes. The shaft 138 is shown to extend into the housing 160 through one of the aforementioned openings with a first end of the shaft 138 terminating in a miter gear 132 and a second end of the shaft 138 configured to be coupled with a power source. The miter gear 132 of the shaft 138 is shown to be positioned adjacent to an additional miter gear 132, with said additional miter gear 132 positioned at an end of a flexible shaft 158. The flexible shaft 158 is shown to extend around the interior geometry of the housing 160 and terminate at an end opposite the miter gear 132 with a worm gear 162. The worm gear 162 is positioned adjacent a bottom portion of the drill bit 128 such that rotation of the shaft 138 is configured to drive rotation of the flexible shaft 158, and thus the worm gear 162, so as to rotate the drill bit 128.

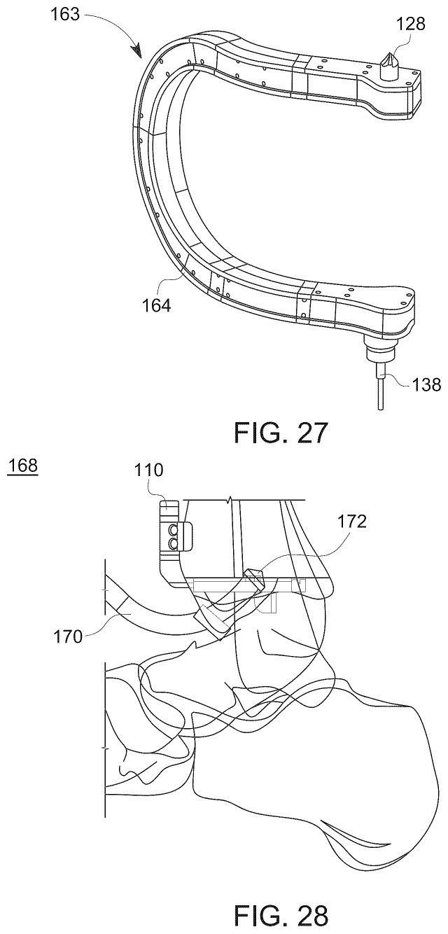

Referring now to FIGS. 26-27, an instrument 163 is shown, according to an exemplary embodiment. The instrument 163 is shown to have a geometry that may be the same as and/or similar to the geometry of the instrument 156, the instrument 126, and/or the instrument 136 as shown and described previously herein. The instrument 163 is shown to include a housing 164, with the housing 164 including an opening at opposite ends thereof. As shown, the first and second openings of the housing 164 are configured about a common longitudinal axis, and it should be understood that such arrangement of first and second openings in the housing 164 may be characteristic of other instruments shown and described herein, for example the instruments 126, 136, etc. However, such arrangement of the first and second openings of the housing may also be about independent longitudinal axes. The shaft 138 is shown to extend into the housing 164 through one of the aforementioned openings with a first end of the shaft 138 terminating in a miter gear 132 and a second end of the shaft 138 configured to be coupled with a power source. The miter gear 132 of the shaft 138 is shown to be positioned adjacent to an additional miter gear 132, with said additional miter gear 132 positioned at an end of a flexible shaft 160. The flexible shaft 160 is shown to extend around the interior geometry of the housing 164 and terminate at an end opposite the miter gear 132 with the worm gear 162. In some aspects, the flexible shaft 166 may be the same as the flexible shaft 158, or may be different than the flexible shaft 158 in that the flexible shaft 166 may be configured to accommodate a U-shaped housing, whereas the flexible shaft 158 may be configured to accommodate a C-shaped housing. The worm gear 162 is positioned adjacent a bottom portion of the drill bit 128 such that rotation of the shaft 138 is configured to drive rotation of the flexible shaft 166, and thus the worm gear 162, so as to rotate the drill bit 128.

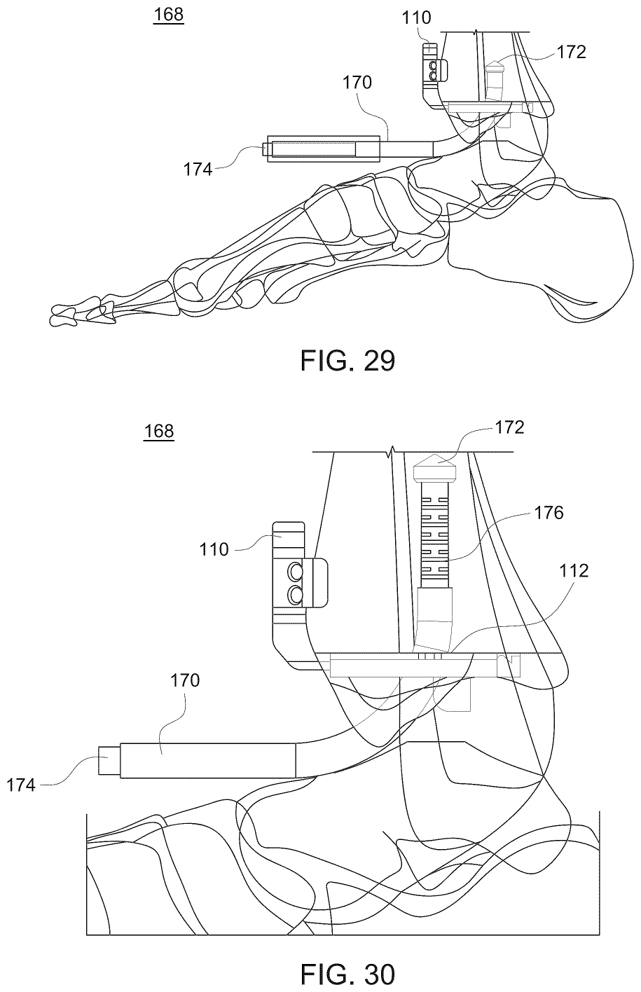

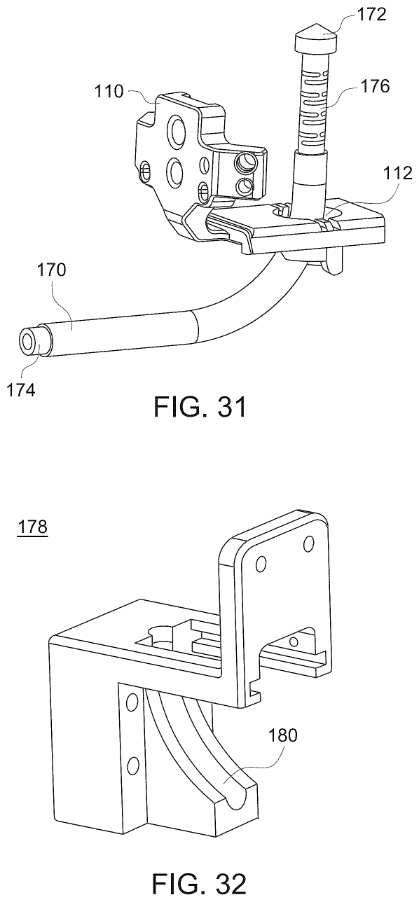

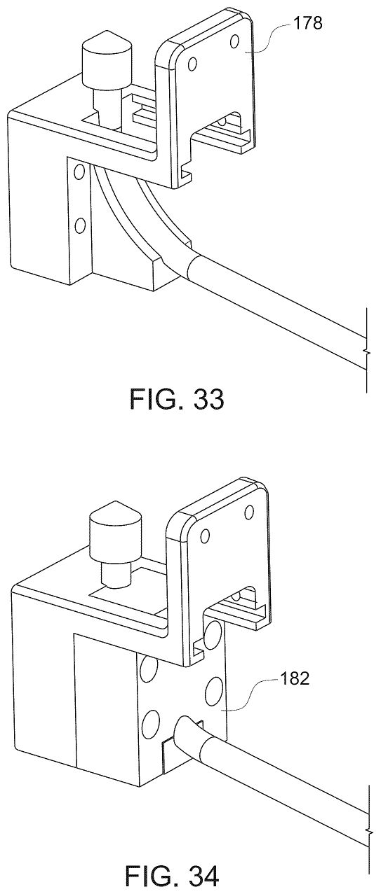

Referring now to FIGS. 28-31, an instrument 168 is shown, according to an exemplary embodiment. The instrument 168 is shown to include a housing 170, which has a substantially non-linear and cylindrical geometry as shown. The instrument 168 is further shown to include a drive shaft 174 which may be coupled or integral with a flexible shaft 176, with said flexible shaft 176 terminating in a drill bit 172 at an end opposite the drive shaft 174. The drive shaft 174 may be couplable to a power source as described previously herein with reference to similar components. In some aspects, the flexible shaft 176 may be flexible in specific directions and/or orientations, for example within a range (degrees, etc.) conducive to manipulation specific to the anatomy involved in specific procedures. The housing 170 may be configured to receive at least a portion of the drive shaft 174, the flexible shaft 176, and/or the drill bit 172 therein so as to guide said component into and through the anterior window between the tibia and talus such that the drill bit 172 may emerge from the housing to contact a portion of the distal tibia (or be positioned adjacent to the distal tibia). In some aspects, the instrument 168 may be implemented in conjunction with the tibial trial 110, there the housing 170 is manipulated by a physician to be adjacent or partially within the opening 112. With reference to FIGS. 31-34, instruments 178 and 182 are shown which may be implemented in conjunction with the instrument 168. The instrument 178 may be configured to releasably couple with the tibial trial 110 so as to guide the instrument 168 (and the housing 170 thereof) along a path 180 defined by an elongated concavity in the instrument 178 (which corresponds to the geometry of the instrument and housing 168, 170). The instrument 182 may be configured to releasably couple with the instrument 178 so as to support the instrument 168 and components thereof as they are advanced along the path 180 and drilling is performed in the distal tibia.

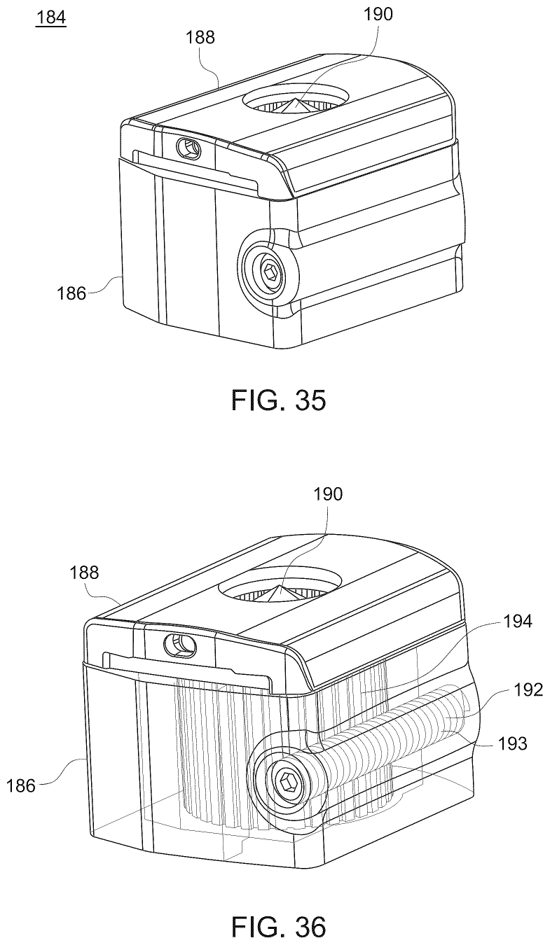

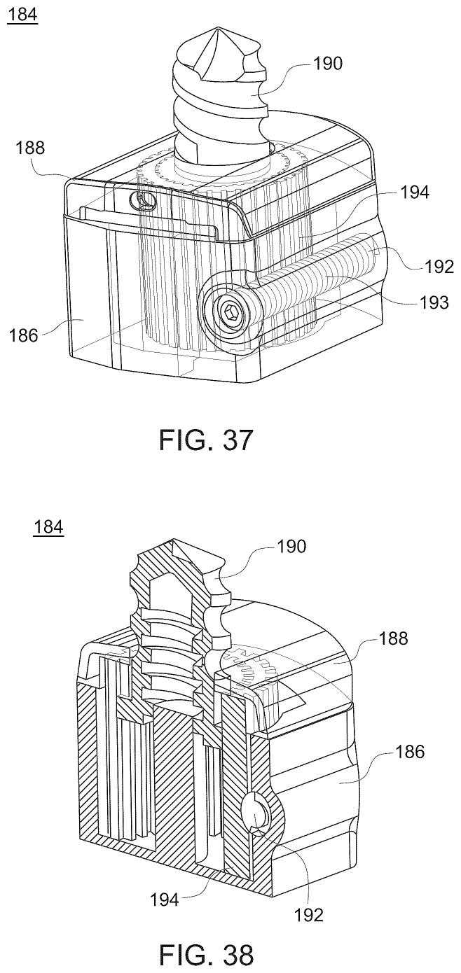

Referring now to FIGS. 35-38, an instrument system 184 (“system 184”) is shown, according to an exemplary embodiment. The system 184 is shown to include a body 186 coupled to a bottom portion of a tibial trial 188, where said body 186 includes at least a portion of a drill bit 190, a drive shaft 192, a worm gear 193, and a spur gear 194. The drive shaft 192 is shown to include the worm gear 193 at a first end thereof, and may be couplable with a power source at an opposite end. Upon actuation (e.g., rotation) of the drive shaft 192, the worm gear 193 engages the spur gear 194 thus rotating the spur gear 194 within the body 186. The spur gear 194 is engaged with the drill bit 190 such that rotation of the spur gear 194 rotates the drill bit 190. The drill bit 190 is configured to be translatable along a vertical axis (similar or the same to that about which it is rotatable) such that at least a portion of the drill bit 190 protrudes through an opening in the tibial trial 188. Accordingly, the system 184 may be placed adjacent the distal tibia (e.g., within the anterior window) with the drive shaft 192 coupled with a power source. Upon actuation of the drive shaft 192, the drill bit 190 is subsequently rotated to drill in an upward direction into the distal tibia.

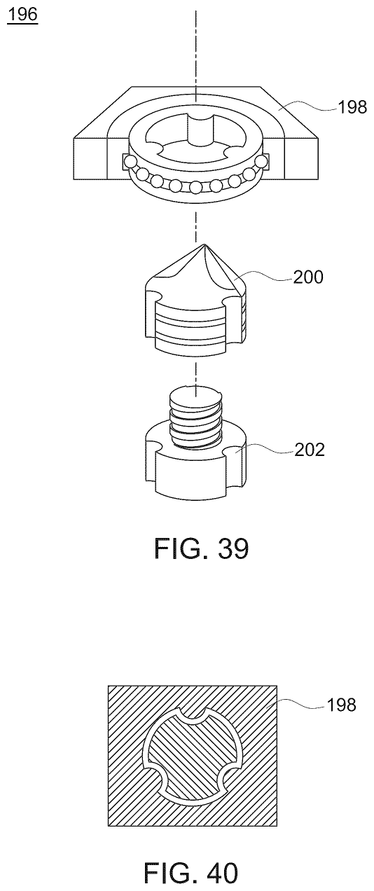

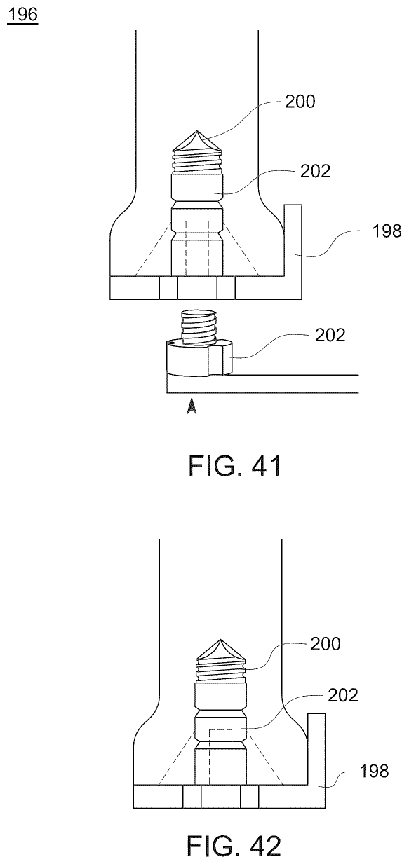

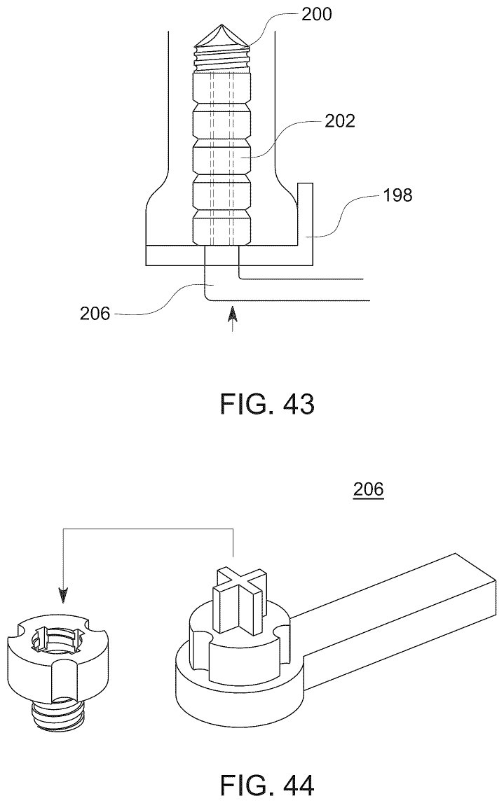

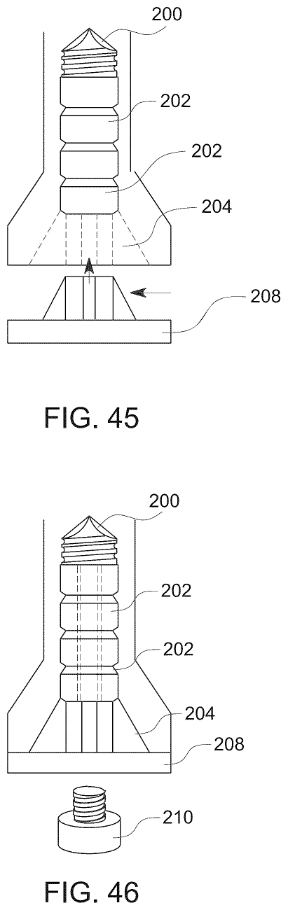

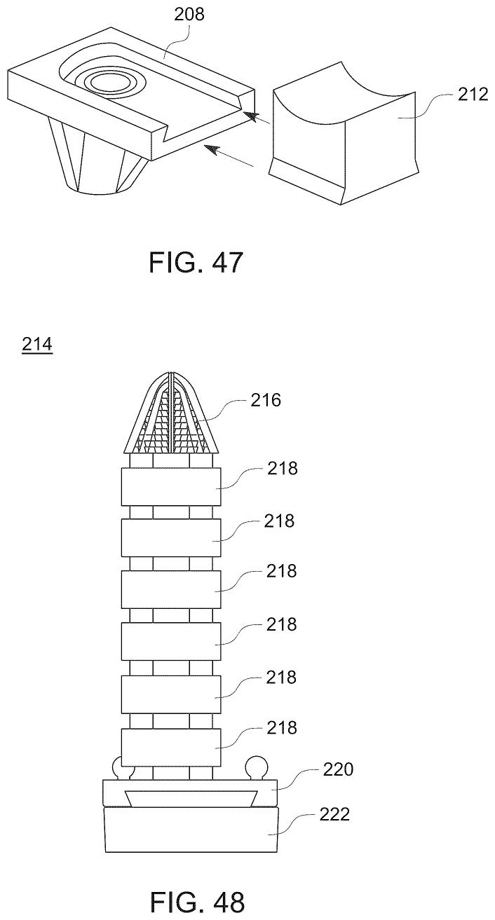

Referring now to FIGS. 39-46, a system 196 (“system 196”) is shown, according to an exemplary embodiment. The system 196 is shown to include a tibial component 198, as well as a drill bit 200 and a modular component 202. The tibial component 198 is shown to include an opening in the central portion thereof through which the drill bit 200 and one or more modular components 202 may be translated and otherwise manipulated. In some aspects, the tibial component may be a tibial trial or a tibial implant component configured to be coupled with or positioned adjacent to the distal tibial while the distal tibia is drilled through the central opening thereof by the drill bit 200. In order to reach more proximal portions of the distal tibia, one or more modular components 202 may be coupled with the drill bit 200 and one another to incrementally increase the height of the drilling apparatus and accordingly, access more proximal portions of the distal tibia. In some aspects, the modular components 202 may include interfaces on at least a portion of a surface thereof configured to interface with an instrument 206 thus facilitating manipulation and drilling. Further, in some aspects the modular components 202 may be configured to lock with one another and ultimately form a portion of a tibial implant (e.g., a stem portion) and also lock with the tibial component 198 (thus eliminating the need for removal after drilling). With reference to FIGS. 45-47, the system 196 is shown with a tibial component 208 configured to be implemented in conjunction with the drill bit 200 and one or more modular components 202. The tibial component 208 is configured to include an engagement feature (e.g., a keel) configured to occupy a corresponding volume 204 (e.g., created by a keel punch such as those shown and described herein). The tibial component 208 is further shown to receive a fastener 210 configured to couple the tibial component 208 with the one or more modular components 202 (and in some aspects, the drill bit 200 which serves as a proximal-most point of the tibial stem). Further, an underside of the tibial component 208 may be configured to releasably couple with an intermediate component 212 of an ankle arthroplasty system (e.g., a poly component).

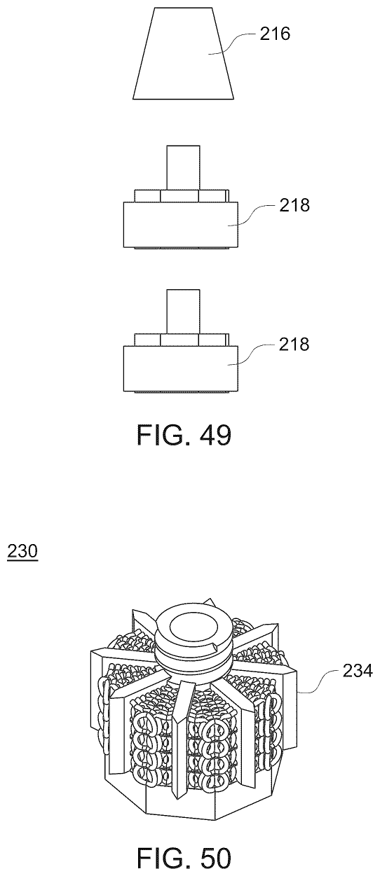

Referring now to FIGS. 48-49, an implant system 214 (“system 214”) is shown, according to an exemplary embodiment. The system 214 is shown to include a tibial component 220, which is configured to releasably couple (via geometries disposed on an underside thereof) with an intermediate (e.g., poly) component 222 as components of a total ankle arthroplasty system. The system 214 is further shown to include one or more (as shown, a plurality) modular components 218 configured to releasably couple with each other in a stacking configuration. In some aspects, each of the modular components 218 may be identical while in other configurations some of the modular components 218 may differ from others. Each of the modular components 218 is shown to include a first interface on a bottom surface thereof and a second interface on a top surface thereof, with the first and second interfaces opposite one another (e.g., first interface being male, second being female, etc.). The tibial component 220 is further shown to be couplable with at least one of the modular components 218 on an upper surface thereof (opposite the coupling with the intermediate component 222). In some aspects, the modular components 218 may be configured to drill and/or cut, while in other aspects the modular components 218 may be configured to be implanted after drilling operations have created a volume in the distal tibia that corresponds to the geometry of the modular components 218. The system 214 is further shown to include a tip 216, which may include a drill bit or other cutting component configured to facilitate forming a volume in the distal tibia. In some aspects, the tip 216 may be implemented in forming the aforementioned volume and remain in said volume while one or more modular components are coupled therewith so as to form a tibial stem of an appropriate length for the volume.

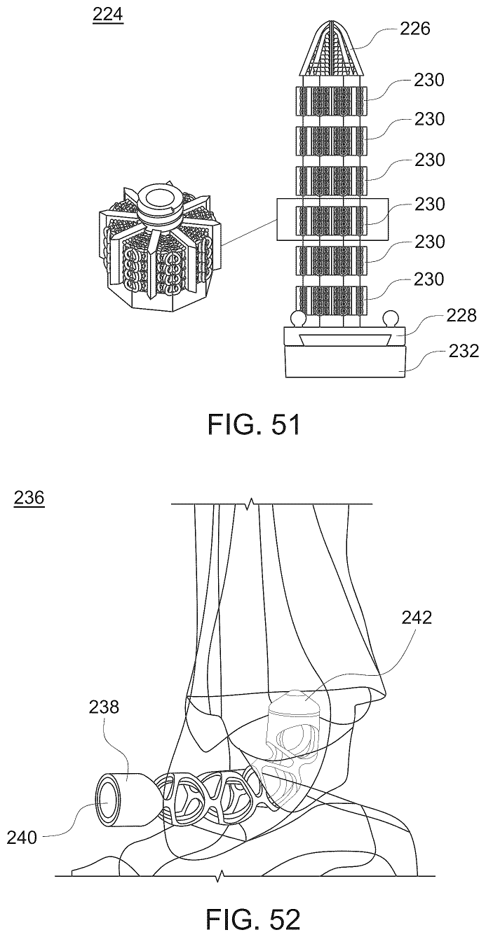

Referring now to FIGS. 50-51, an implant system 224 (“system 224”) is shown, according to an exemplary embodiment. The system 224 is shown to include a tibial component 228, which is configured to releasably couple (via geometries disposed on an underside thereof) with an intermediate (e.g., poly) component 232 as components of a total ankle arthroplasty system. The system 224 is further shown to include one or more (as shown, a plurality) modular components 230 configured to releasably couple with each other in a stacking configuration. In some aspects, each of the modular components 230 may be identical while in other configurations some of the modular components 230 may differ from others. Each of the modular components 230 is shown to include a first interface on a bottom surface thereof and a second interface on a top surface thereof, with the first and second interfaces opposite one another (e.g., first interface being male, second being female, etc.). The tibial component 228 is further shown to be couplable with at least one of the modular components 230 on an upper surface thereof (opposite the coupling with the intermediate component 232). In some aspects, the modular components 230 may be configured to drill and/or cut and may include one or more cutting features 234 the same as or similar to that shown in FIG. 50. The system 224 is further shown to include a tip 226, which may include a drill bit or other cutting component configured to facilitate forming a volume in the distal tibia. In some aspects, the tip 216 may be implemented in forming the aforementioned volume and remain in said volume while one or more modular components are coupled therewith so as to advance the tip further proximally in the distal tibia.

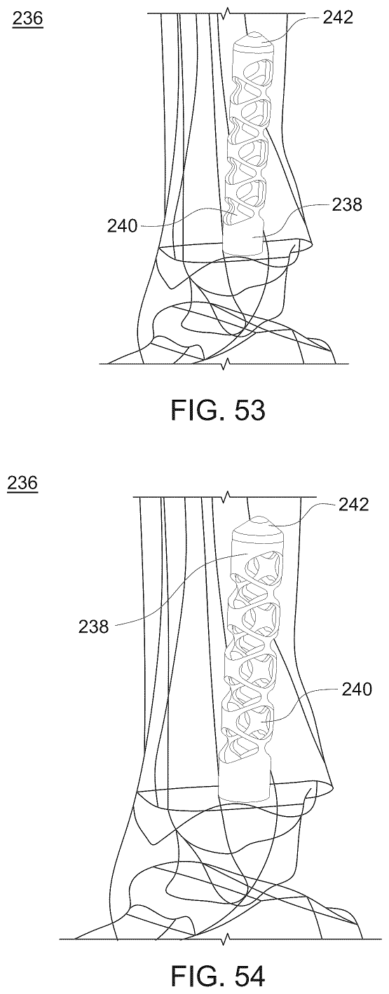

Referring now to FIGS. 52-54, an implant system 236 is shown, according to an exemplary embodiment. The implant system 236 (“system 236”) may include one or more components the same as and/or similar to that of the system 168 as shown and described previously herein. The system 236 is shown to include an outer component 238 and an inner component 240, with said inner component 240 including a tip 242 at a terminal end thereof which may be implemented in drilling or otherwise forming a volume in the distal tibia. In some aspects, both the outer and inner components 238, 240 may be flexible (e.g., bendable to a substantially 90-degree angle or other bend angles specific to anatomy adjacent to or involved in a specific procedure, or bendable about a specific radius of curvature or range of radii, etc.) about a specific plane, but rigid (e.g., not bendable) about an orthogonal plane. Accordingly, the outer and inner components 238, 240 may be positioned such that the planes about which each are flexible align and the components 238, 240 bent to approximately 90-degrees and inserted in a volume in the distal tibia as shown in FIG. 52. Then, once positioned as desired within the volume, one or both components 238, 240 may be rotated so as to configure the planes about which each component is flexible at orthogonal angles relative to another. Accordingly, in such an orientation the outer component 238 may be prevented from bending about its flexible plane by alignment of a rigid plane of the inner component 240 with said flexible plane, and vice versa. In some embodiments, one or both of the components 238, 240 may be bendable/flexible about multiple planes or an infinite amount of planes. One or both of the outer and inner components 238, 240 may include geometries at a distal-most end (when implanted as shown; opposite the tip 242) configured to facilitate coupling with a tibial tray or other similar component of a tibial implant. Further, the system 224 may be implemented in conjunction with other implants and/or implant systems, for example ankle arthroplasty systems.

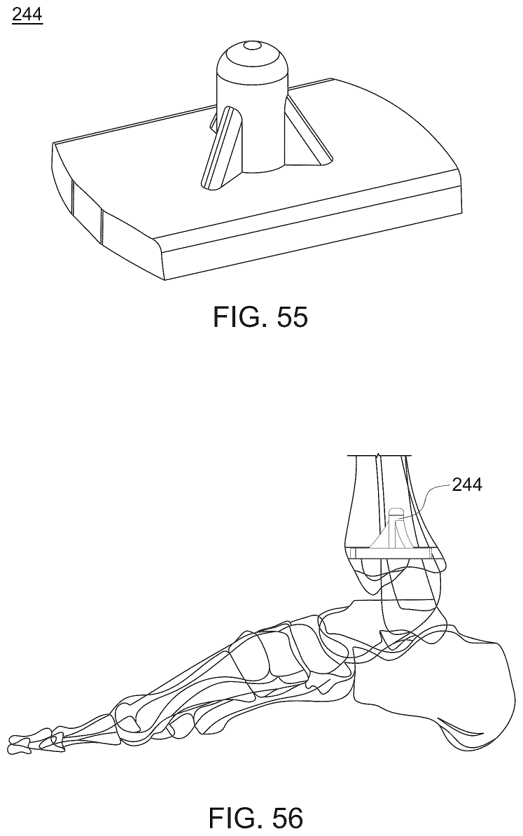

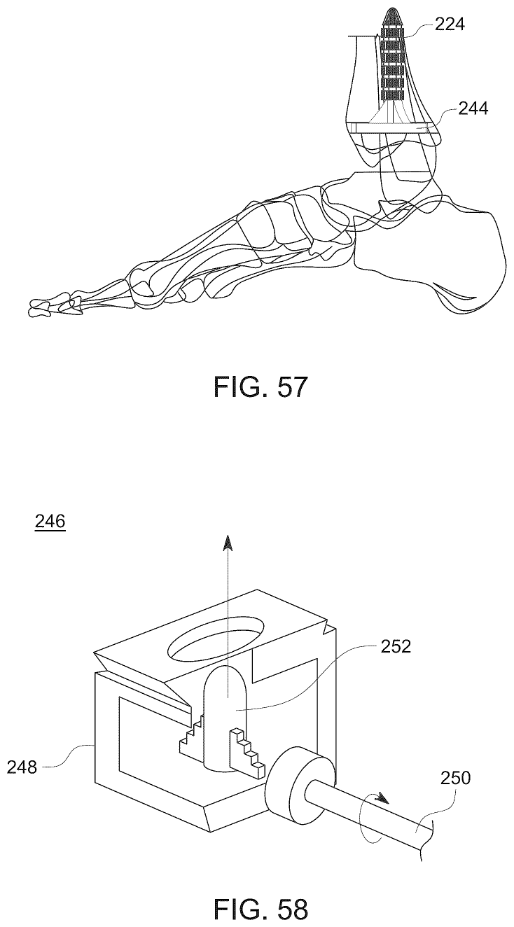

Referring now to FIGS. 55-57, an implant 244 is shown, according to an exemplary embodiment. The implant 244 is shown to be a tibial implant with a keel feature on an upper surface thereof. As shown, the keel feature includes a central element (e.g., a post, etc.) about which each of the keels are incrementally spaced. In some aspects, the implant 244 may include greater or fewer keels than shown, for example two to eight keels. Further, in some aspects said keels may be equally spaced from one another, while in some aspects said keels may be at least partially unevenly spaced from one another. In some aspects, such as that shown in FIG. 57, the implant 244 may be implemented in conjunction with one or more other implant systems/components, for example implant system 224 and/or components thereof. Accordingly, in some aspects, the implant 244 may include one or more coupling features disposed thereon to facilitate coupling with one or more other implant systems and/or components thereof.

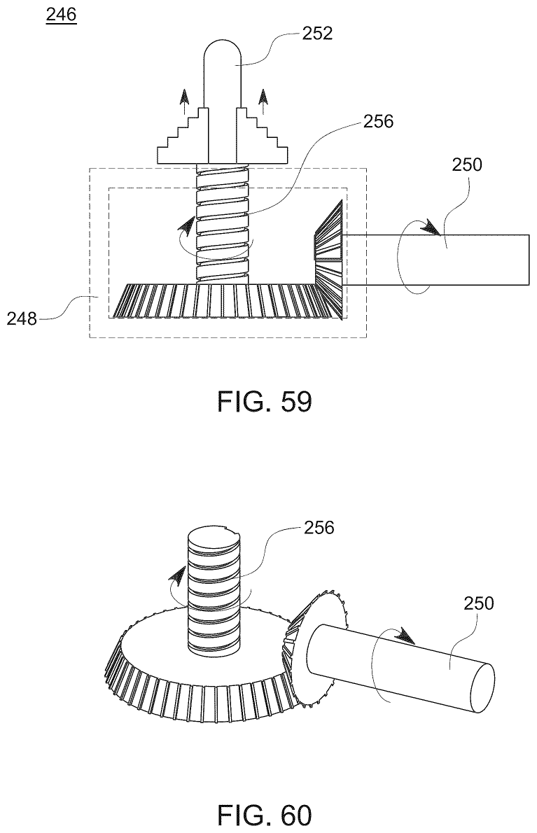

Referring now to FIGS. 58-60, an instrument system 246 (“system 246”) is shown, according to an exemplary embodiment. The system 246 is shown to include a housing 248, with said housing including at least a portion of a first geared member 250 and a second geared member 256 therein. The first geared member 250 is shown to include a drive shaft configured to couple with a power source at a first end and terminate with a miter gear at an opposite, second end. The miter gear is configured to engage with a complimentary miter gear at a first end of the second gear mechanism, which is positioned opposite a threaded shaft from coupling with a keel punch 252. Accordingly, upon actuation of the first geared member 250 (e.g., rotation driven by a power source), the second geared member 256 is rotated and thus translates the keel punch 252 (which may translate along the threading of the threaded shaft) in an upward direction such that the keel punch 252 protruded from an opening in an upper surface of the housing 248 and, when the housing is disposed within the anterior window, contacts the distal portion of the tibia.

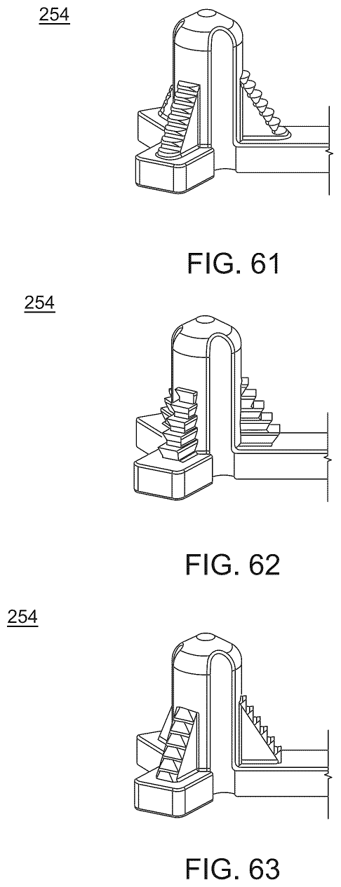

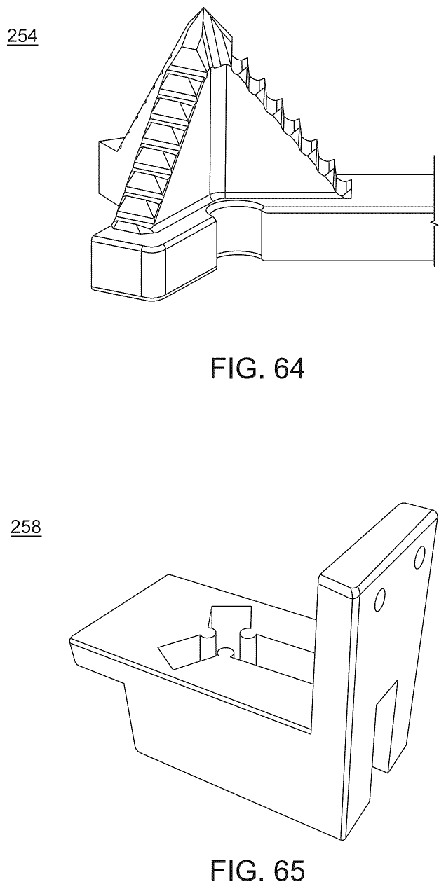

Referring now to FIGS. 61-64, an instrument 254 is shown. The instrument 254 is shown to include a keel punch at a terminal end thereof, and, as shown, the keel punch may include various keel sizes and/or geometries. For example, the keel punch may include various quantities of keels, for example between two and five keels (inclusive), and further, may include geometries on one or more surfaces of the keels (e.g., serrations). In some aspects, the keels may be spaced about a central post or other structural element, or in other aspects, the keels may abut one another to form a pointed apex at a central portion thereof.

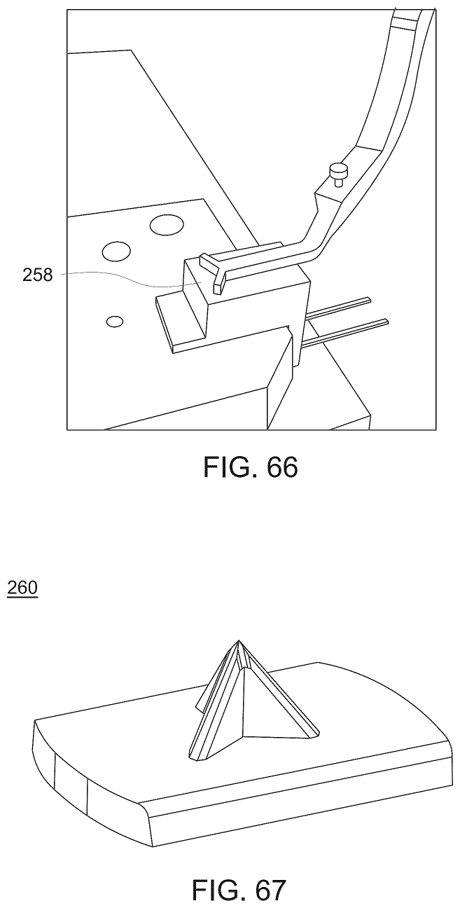

Referring now to FIGS. 65-66, instrument 258 is shown according to an exemplary embodiment. The instrument 258 is shown to include a volume disposed in a central portion of, wherein said volume corresponds to that of a keel punch. Further, a keel punch, or at least a portion thereof, may be configured to be received within the opening of the instrument 258. In some aspects, the instrument 258 may be implemented in conjunction with other instruments, instrument systems, implants, or implant systems shown and described previously and subsequently herein in order to position, guide, or otherwise engage with a keel punch or other similar instrument (or a portion thereof).

Referring now to FIG. 67, an implant 260 is shown, according to an exemplary embodiment. The implant 260 is shown to be a tibial implant with a keel disposed in a central portion thereof. In some aspects, the keel of the implant 260 may have a geometry that corresponds to the geometry of a keel punch or other similar instrument included in a system (e.g., the instrument 254). The implant 260 may be implemented in conjunction with other implant components as part of an implant system, for example tibial implant components configured to couple with the implant 260 and/or other implants, for example talar implants or intermediate components as part of an ankle arthroplasty system.

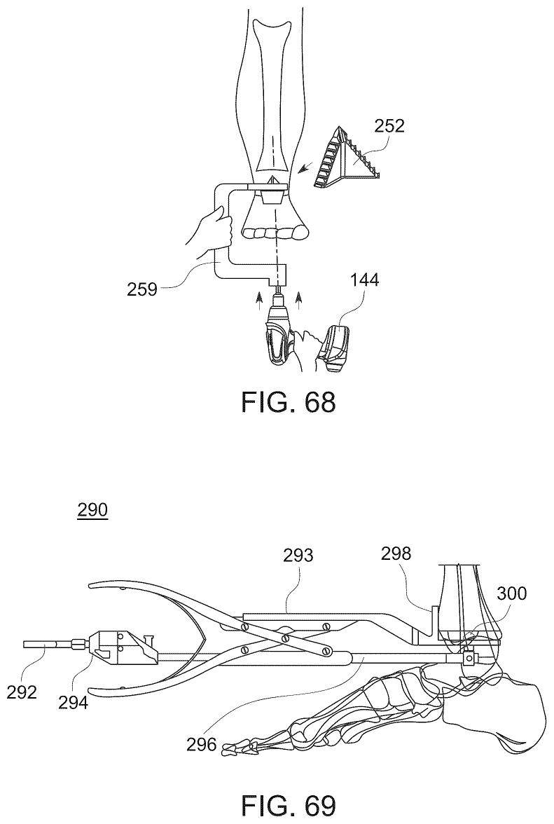

Referring now to FIG. 68, the keel punch 252 is shown to be releasably coupled with an instrument 259, where the instrument 259 may be the same as and/or similar to that shown in FIG. 22 as shown and described previously herein. The instrument 259 is shown to be releasably coupled with (e.g., engaged with, etc.) the power source 144. In some aspects, the power source 144 may be a drill or other rotational element. However, in some aspects, for example, that shown in FIG. 68, the power source 144 may be an impact driver, impact hammer drill, or similar instrument. Further, in aspects such as that of FIG. 68 where the keel punch 252 is implemented, the instrument 259 may be engaged with an impact-based power source 144 configured to drive or “punch” the keel punch 252 into the distal tibia (as opposed to a rotation-based power source 144).

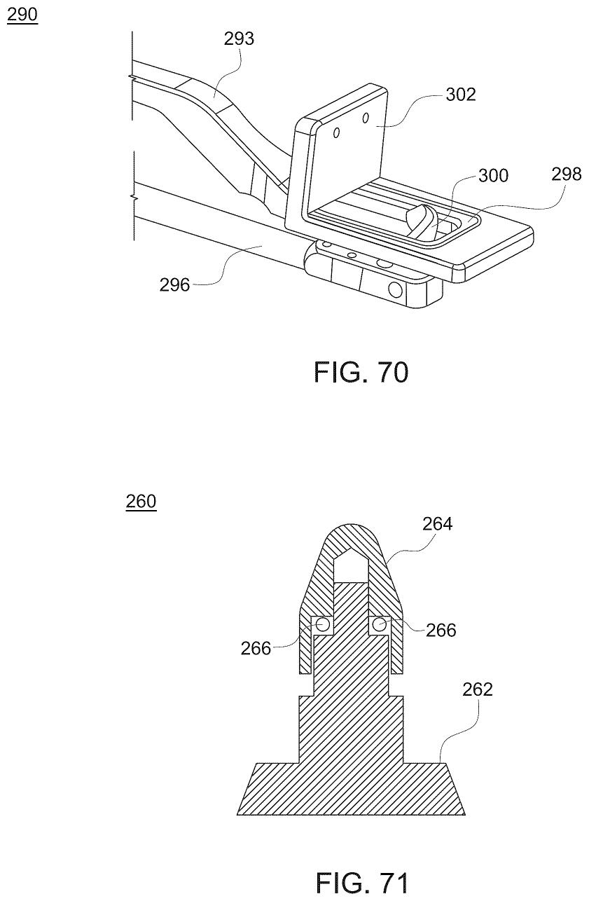

Referring now to FIGS. 69-70, an instrument system 290 (“system 290”) is shown, according to an exemplary embodiment. The system 290 is shown to include a distractor 293, where said distractor 293 has been modified to receive and/or translate a force therethrough provided by a power source (e.g., the power source 144 as shown and described previously herein). In some aspects, the distractor 293 may be configured such that actuation of the distractor 293 by a physician manipulates one or more components at a distal end thereof toward one another (e.g., to retract one portion of a component within another, etc.) The distractor 293 is shown to include a first shaft 292, which may be configured to couple and/or engage with the power source, extending from between handles of the distractor 293 at a proximal end. The first shaft 292 is shown to releasably couple with a modulation element 294 disposed substantially between and/or adjacent the handles of the distractor 293. The modulation element 294 may be configured to translate force/power provided by the power source (e.g., “reduce” or “increase” a gear such that other elements of the system 290 may run at higher or lower speeds, respectively) and, further, may be coupled with a drive shaft 296. The drive shaft 296 is shown to extend from the distractor 293 in a direction substantially opposite that of the first shaft 292. As shown in FIG. 69, the drive shaft 296 extends toward and has been positioned adjacent/within the anterior window at a lower portion thereof. In some aspects, the drive shaft 296 may include one or more gears at a distal portion thereof, for example one or more worm gears and/or spur gears (e.g., similar to that shown in FIG. 1). The drive shaft 296 is shown to be coupled with a drill bit 300 extending upward from a terminal end of the drive shaft 296 and positioned adjacent the distal tibia. The distractor 293 is also shown to include a tibial trial 302 disposed on an upper portion of an end opposite that in which the first shaft 292 extends. Similar to other tibial trials shown and described herein, the tibia trial 302 may include a central opening configured to receive at least a portion of the drill bit 300 or other instrument therethrough as the instrument contacts the distal tibia. Accordingly, a physician may apply a power source to the first shaft 292, with the power source applying a force that ultimately reaches the drill bit 300 (via the modulation element 294 and drive shaft 296) so as to rotate the drill bit 300. As the drill bit 300 rotates, a physician may manipulate the handles of the distractor 293 toward one another such that the drill bit 300 engages and drills into the distal tibia of a patient.

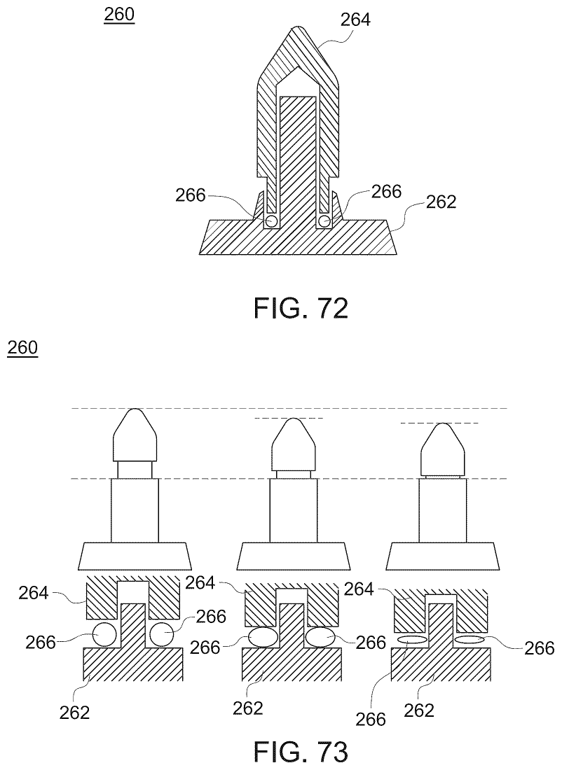

Referring now to FIGS. 71-73, various embodiments of an implant system 260 are shown, according to an exemplary embodiment. The implant system 260 is shown to include a lower portion 262 (e.g., a tibial base plate) and an upper portion 264. The upper portion 264 is shown to define a volume that corresponds to a geometry of the lower portion 262, for example an upward projection of the lower portion 262. The implant system 260 is further shown to include at least one resilient element 266 (e.g., multiple components, a ring/washer, etc.) configured to have dampening properties. The at least one resilient element 266 is configured to be positioned between shoulders of the lower portion 262 and a corresponding geometry of the upper portion 264 so as to dampen forces applied to either implant portion and prevent the upper portion 264 from contacting the lower portion 262 (and vice versa). In some aspects, one or more components of the upper portion 264 may be translatable in response to an applied force such that the resilient element 266 is compressed.

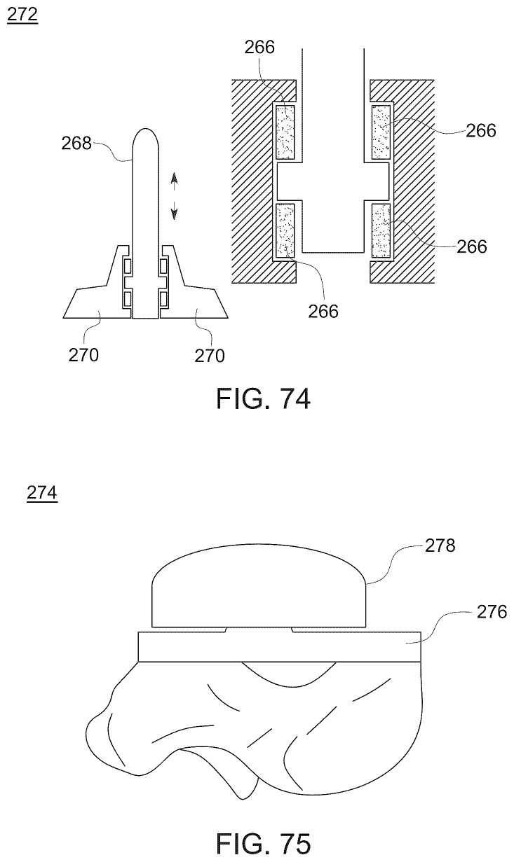

Referring now to FIG. 74, an implant system 272 is shown, according to an exemplary embodiment. The implant system 272 is shown to include a base component 270 (e.g., a tibial base plate) and an inner component 268. The base component 270 is shown to define a volume that corresponds to a geometry of the inner component 268, for example a lateral projection of the inner component 268. The implant system 272 is further shown to include at least one resilient element 266 (e.g., multiple components, rings/washers, etc.) configured to have dampening properties. The at least one resilient element 266 is configured to be positioned between shoulders of the protrusion of the inner component 268 and a corresponding geometry of the base component 270 so as to dampen forces applied to either implant portion and prevent the inner component 268 from contacting the lower base component (and vice versa) should either component be translated up or down in response to an applied force. In some aspects, one or more components of the inner component 268 may be translatable in response to an applied force such that the resilient element 266 is compressed.

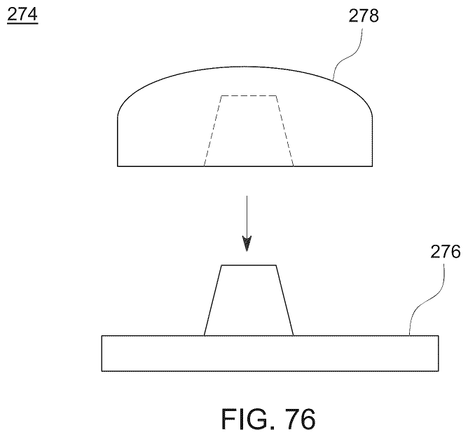

Referring now to FIGS. 75-76, an implant system 274 is shown, according to an exemplary embodiment. The implant system 274, which is a talar implant system, may be implemented in conjunction with various instrument systems and surgical methods, as well as various tibial implant systems (including but not limited to those shown, described, and incorporated by reference herein). The implant system 274 is shown to include a talar base 276 and a modular component 278, with the modular component 278 configured to releasably couple or to be positioned adjacent the talar base 276. In some aspects, the implant system 274 may be provided to a physician with multiple embodiments of the modular component 278. For example, the implant system 274 may be provided with various sizes of the modular component 278, or may be provided with various modular components 278 having varying geometries and/or other structural features or characteristics. In some aspects, the modular component 278 and/or the talar base 276 may be patient specific (e.g., created custom to the geometry of the anatomy of a patient). The components of the implant system 274 may also be configured to interface with adjacent anatomy and/or surfaces of other implants that may be positioned within a patient adjacent to the implant system 274. Further, in some aspects the modular component 278 may be configured to rotate internally/externally relative to the talar base 276 and/or other components that may be implemented in conjunction with the implant system 274.

Given the illustrated examples above, the present disclosure provides in one aspect an instrument 100, 126, 156, 163, 184, comprising: a housing 106, 130, 160, 164, 186 comprising a first end and a second end, a first drive shaft 104, 134, 158, 166, 192 received at least partially within the housing 106, 130, 160, 164, 186, a first gear member 105, 129, 162, 193 positioned at a first end of the first drive shaft 104, 134, 158, 166, 192, a second gear member 107, 194 disposed within the first end of the housing 106, 130, 160, 164, 186, wherein the second gear member 107, 194 engages the first gear member 105, 129, 162, 193, and a drill bit 108, 128, 190 engaging the second gear member 107, 194 and extending out from at least a portion of the housing 106, 130, 160, 164, 186.

In some embodiments, the first gear member 105, 129, 162, 193 is positioned adjacent to the second gear member 107, 194.

In some embodiments, the housing 106, 130, 160, 164, 186 further includes a first opening positioned at the first end and a second opening positioned at the second end. In some embodiments, the first opening is positioned perpendicular to the second opening. In some embodiments, the first drive shaft 104, 134, 158, 166, 192 extends through at least a portion of the first opening and the drill bit 108, 128, 190 extends through the second opening.

In some embodiments, the first gear member 105, 129, 162, 193 rotatably engages the second gear member 107, 194 and the coupled drill bit 108, 128, 190.

In some embodiments, the instrument 100, 126, 156, 163, 184 of claim 1, further comprises an outer shaft 102 coupled to the housing 106, 130, 160, 164, 186 on a first end and surrounding at least a portion of the first drive shaft 104, 134, 158, 166, 192.

In some embodiments, the drill bit 108, 128, 190 is integral with or coupled with an upper surface of the second gear member 107, 194.

In some embodiments, the instrument 100, 126, 156, 163, 184 further comprises a drill 144 coupled to a second end of the first drive shaft 104, 134, 158, 166, 192.

In some embodiments, the first gear member 105, 129, 162, 193 is integral with the first drive shaft 104, 134, 158, 166, 192.

In some embodiments, the drill bit 108, 128, 190 is at least one of integral with or coupled with an upper surface of the second gear member 107, 194.

In some embodiments, the instrument 100, 126, 156, 163, 184 further comprises one or more modular components 114, wherein the one or more modular components 114 are releasably coupled to the drill bit 108, 128, 190 on a first end and the second gear member 107, 194 on a second end.

In some embodiments, the instrument 100, 126, 156, 163, 184 further comprises a distractor 116, wherein a portion of the distractor 116 engages a portion of the first drive shaft 104, 134, 158, 166, 192 to vertically translate the first drive shaft 104, 134, 158, 166, 192 relative to the distractor 116.

In some embodiments, the instrument 100, 126, 156, 163, 184 further comprises a second drive shaft 134 with a third gear member 132 positioned on a first end of the second drive shaft 134, wherein the third gear member 132 rotatably engages the first gear member 105, 129, 162, 193.

In some embodiments, the instrument 100, 126, 156, 163, 184 further comprises a drill 144 rotatably coupled to a second end of the second drive shaft 134.

In another aspect, the present disclosure provides an instrument system, comprising an instrument 100, 126, 156, 163, 184 and a tibial trial 110 with at least one opening 112, wherein the drill bit 108, 128, 190 of the instrument 100 is configured to rotatably pass through the at least one opening 112. The instrument 100, 126, 156, 163, 184 comprising a housing 106, 130, 160, 164, 186 with a first end and a second end, a first drive shaft 104, 134, 158, 166, 192 received at least partially within the housing 106, 130, 160, 164, 186, a first gear member 105, 129, 162, 193 positioned at a first end of the first drive shaft 104, 134, 158, 166, 192, a second gear member 107, 194 disposed within the first end of the housing 106, 130, 160, 164, 186, wherein the second gear member 107, 194 engages the first gear member 105, 129, 162, 193, and a drill bit 108, 128, 190 engaging the second gear member 107, 194 and extending out from at least a portion of the housing 106, 130, 160, 164, 186.

In some embodiments, the instrument system further comprises a tibial implant. In some embodiments, the at least one opening 112 of the tibial trial 110 corresponds to a position of at least one attachment means of the tibial implant.

In some embodiments, the at least one opening 112 is two openings 112.

In a further aspect, the present disclosure provides a method of using an instrument 100, 126, 156, 163, 184, comprising obtaining an instrument 100, 126, 156, 163, 184, performing a surgical procedure to position a tibial trial 110 onto a tibia, inserting the instrument 100, 126, 156, 163, 184 such that a drill bit 108, 128, 190 is aligned with at least one opening in the tibial trial 110, using a drill 144 to rotate the drill bit 108, 128, 190, applying a vertical translating force to at least a portion of the instrument 100, 126, 156, 163, 184 to drill at least one opening into the tibia, and removing the drill bit 108, 128, 190 from the tibia and the instrument 100, 126, 156, 163, 184 from the patient.

In some embodiments, the instrument 100, 126, 156, 163, 184, comprises a housing 106, 130, 160, 164, 186 comprising a first end and a second end, a first drive shaft 104, 134, 158, 166, 192 received at least partially within the housing 106, 130, 160, 164, 186, a first gear member 105, 129, 162, 193 positioned at a first end of the first drive shaft 104, 134, 158, 166, 192, a second gear member 107, 194 disposed within the first end of the housing 106, 130, 160, 164, 186, wherein the second gear member 107, 194 engages the first gear member 105, 129, 162, 193, and a drill bit 108, 128, 190 engaging the second gear member 107, 194 and extending out from at least a portion of the housing 106, 130, 160, 164, 186.

In some embodiments, the at least one opening in the tibial trial 110 is two openings and wherein the at least one opening drilled into the tibial is two openings.

In some embodiments, the vertical translating force is applied to the instrument 100, 126, 156, 163, 184 by a distractor 116.

In some embodiments, using a drill 144 to rotate the drill bit 108, 128, 190 comprises activating the drill 144 to rotate the first drive shaft 104, 134, 158, 166, 192 and the coupled first gear member 105, 129, 162, 193. In some embodiments, the first gear member 105, 129, 162, 193 engages and rotates the second gear member 107, 194, and wherein the rotating of the second gear member 107, 194 rotates the drill bit 108, 128, 190.

In some embodiments, the instrument 100, 126, 156, 163, 184 of claim 1, further comprises a second drive shaft 134 with a third gear member 132 positioned on a first end of the second drive shaft 134, wherein the third gear member 132 rotatably engages the first gear member 105, 129, 162, 193. In some embodiments, using a drill 144 to rotate the drill bit 108, 128, 190 comprises activating the drill 144 to rotate the second drive shaft 134 and the coupled third gear member 132. In some embodiments, the third gear member 132 rotates the first drive shaft 104, 134, 158, 166, 192 and the coupled first gear member 105, 129, 162, 193. In some embodiments, the first gear member 105, 129, 162, 193 engages and rotates the second gear member 107, 194, and wherein the rotating of the second gear member 107, 194 rotates the drill bit 108, 128, 190.

In still a further aspect, the present disclosure provides an instrument, comprising a housing comprising a first end, a second end, and a body disposed therebetween. In some embodiments, the housing further comprises a first opening positioned at the first end and a second opening positioned at the second end. In some embodiments, the first opening and the second opening are positioned about a common longitudinal axis. In some embodiments, at least a portion of the body of the housing is positioned substantially parallel to the common longitudinal axis. In some embodiments, the instrument further comprises a first drive shaft received at least partially within the first opening and extending into the housing. In some embodiments, at least one end of the first drive shaft terminates in a first gear member. In some embodiments, the instrument further comprises a second drive shaft disposed at least partially within the housing. In some embodiments, the second drive shaft comprises a second gear member disposed at a first end of the second drive shaft such that the second gear member engages the first gear member and a third gear member disposed at a second end of the second drive shaft opposite the first end of the second drive shaft. In some embodiments, the first drive shaft comprises a substantially linear geometry and the second drive shaft comprises a substantially non-linear geometry. In some embodiments, the instrument further comprises an engagement member extending at least partially from the second opening of the housing and positioned such that the engagement member is engaged by the third gear member.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. An instrument, comprising:
a housing comprising a first end and a second end;
a first drive shaft received at least partially within the housing;
a first gear member positioned at a first end of the first drive shaft;
a second gear member disposed within the first end of the housing, wherein the second gear member engages the first gear member; and
a drill bit engaging the second gear member and extending out from at least a portion of the housing.

2. The instrument of claim 1, wherein the first gear member is positioned adjacent to the second gear member.

3. The instrument of claim 1, wherein the housing further comprises:
a first opening positioned at the first end; and
a second opening positioned at the second end,
wherein the first opening is positioned perpendicular to the second opening,
wherein the first drive shaft extends through at least a portion of the first opening, and
wherein the drill bit extends through the second opening.

4. The instrument of claim 1, wherein the first gear member rotatably engages the second gear member and the coupled drill bit.

5. The instrument of claim 1, further comprising:
an outer shaft coupled to the housing on a first end and surrounding at least a portion of the first drive shaft.

6. The instrument of claim 1, wherein the drill bit is integral with or coupled with an upper surface of the second gear member.

7. The instrument of claim 1, further comprising:
a drill coupled to a second end of the first drive shaft.

8. The instrument of claim 1, wherein the first gear member is integral with the first drive shaft.

9. The instrument of claim 1, wherein the drill bit is at least one of integral with or coupled with an upper surface of the second gear member.

10. The instrument of claim 1, further comprising:
one or more modular components, wherein the one or more modular components are releasably coupled to the drill bit on a first end and the second gear member on a second end.

11. The instrument of claim 1, further comprising:
a distractor, wherein a portion of the distractor engages a portion of the first drive shaft to vertically translate the first drive shaft relative to the distractor.

12. The instrument of claim 1, further comprising:
a second drive shaft with a third gear member positioned on a first end of the second drive shaft, wherein the third gear member rotatably engages the first gear member.

13. The instrument of claim 12, further comprising:
a drill rotatably coupled to a second end of the second drive shaft.

14. An instrument system, comprising:
an instrument, comprising:
a housing with a first end and a second end;
a first drive shaft received at least partially within the housing;
a first gear member positioned at a first end of the first drive shaft;
a second gear member disposed within the first end of the housing,
wherein the second gear member engages the first gear member; and
a drill bit engaging the second gear member and extending out from at least a portion of the housing; and
a tibial trial with at least one opening, wherein the drill bit of the instrument is configured to rotatably pass through the at least one opening.

15. The instrument system of claim 14, further comprising:
a tibial implant, wherein the at least one opening of the tibial trial corresponds to a position of at least one attachment means of the tibial implant.

16. The instrument system of claim 14, wherein the at least one opening is two openings.

17. A method of using an instrument, comprising:
obtaining an instrument;
performing a surgical procedure to position a tibial trial onto a tibia;
inserting the instrument such that a drill bit is aligned with at least one opening in the tibial trial;
using a drill to rotate the drill bit;
applying a vertical translating force to at least a portion of the instrument to drill at least one opening into the tibia; and
removing the drill bit from the tibia and the instrument from the patient.

18. The method of claim 17, wherein the instrument comprises:
a housing comprising a first end and a second end;
a first drive shaft received at least partially within the housing;
a first gear member positioned at a first end of the first drive shaft;
a second gear member disposed within the first end of the housing, wherein the second gear member engages the first gear member; and
a drill bit engaging the second gear member and extending out from at least a portion of the housing.

19. The method of claim 17, wherein the at least one opening in the tibial trial is two openings and wherein the at least one opening drilled into the tibial is two openings, and
wherein the vertical translating force is applied to the instrument by a distractor.

20. The method of claim 18, wherein using a drill to rotate the drill bit comprises:
activating the drill to rotate the first drive shaft and the coupled first gear member;
wherein the first gear member engages and rotates the second gear member, and wherein the rotating of the second gear member rotates the drill bit.
